(19) United States
(12) Patent Application Publication
(10) Pub. No.: US 2007/0093787 A1
(43) Pub. Date: Apr. 26, 2007

(54) IONTOPHORESIS DEVICE TO DELIVER MULTIPLE ACTIVE AGENTS TO BIOLOGICAL INTERFACES

(75) Inventor: Gregory A. Smith, Union City, CA (US)

Correspondence Address: SEED INTELLECTUAL PROPERTY LAW GROUP PLLC, 701 FIFTH AVE, SUITE 5400, SEATTLE, WA, 98104, US

(73) Assignee: Transcutaneous Technologies Inc., Tokyo (JP)

(21) Appl. No.: 11/537,006

(22) Filed: Sep. 29, 2006

Related U.S. Application Data
(60) Provisional application No. 60/722,674, filed on Sep. 30, 2005.

(51) Int. Cl. A61K 9/22

(52) U.S. Cl. 604/890.1

(57) ABSTRACT

An iontophoresis device includes active and counter electrode assemblies. The active electrode assembly includes an active electrode element and at least two laterally spaced active agent reservoirs. The active electrode assembly may also include an outermost ion selective membrane caching an active agent and a further active agent carried by an outer surface of the outermost ion selective membrane. The active electrode assembly may also include an electrolyte reservoir storing electrolyte and an inner ion selective membrane positioned between the electrolyte reservoir and the active agents. The active electrode may also include an inner withdrawable sealing liner between the electrolyte reservoir and the active agents. An outer release liner may protectively cover or overlay the further active agent and/or outer surface prior to use. The active electrode assembly may also include a blister pack of at least two hydrating agent blisters to selectively hydrate dehydrated active agent.

### CROSS-REFERENCE TO RELATED APPLICATION

This application claims the benefit under 35 U.S.C. §119(e) of U.S. Provisional Patent Application No. 60/722,674 filed Sep. 30, 2005, where this provisional application is incorporated herein by reference in its entirety.

### BACKGROUND OF THE INVENTION

1. Field of the Invention

This disclosure generally relates to the field of iontophoresis, and more particularly to the effective delivery of active agents such as therapeutic agents or drugs to a biological interface under the influence of electromotive force.

2. Description of the Related Art

Iontophoresis employs an electromotive force and/or current to transfer an active agent (e.g., a charged substance, an ionized compound, an ionic a drug, a therapeutic, a bioactive-agent, and the like), to a biological interface (e.g., skin, mucus membrane, and the like), by applying an electrical potential to an electrode proximate an iontophoretic chamber containing a similarly charged active agent and/or its vehicle.

Iontophoresis devices typically include an active electrode assembly and a counter electrode assembly, each coupled to opposite poles or terminals of a power source, for example a chemical battery or an external power source. Each electrode assembly typically includes a respective electrode element to apply an electromotive force and/or current. Such electrode elements often comprise a sacrificial element or compound, for example silver or silver chloride. The active agent may be either cationic or anionic, and the power source may be configured to apply the appropriate voltage polarity based on the polarity of the active agent. Iontophoresis may be advantageously used to enhance or control the delivery rate of the active agent. The active agent may be stored in a reservoir such as a cavity. See e.g., U.S. Pat. No. 5,395,310. Alternatively, the active agent may be stored in a reservoir such as a porous structure or a gel. An ion exchange membrane may be positioned to serve as a polarity selective barrier between the active agent reservoir and the biological interface. The membrane, typically only permeable with respect to one particular type of ion (e.g., a charged active agent), prevents the back flux of the oppositely charged ions from the skin or mucous membrane.

Commercial acceptance of iontophoresis devices is dependent on a variety of factors, such as cost to manufacture, shelf-life or stability during storage, efficiency of active agent delivery, safety of operation, and disposal issues.

Proper treatment and/or diagnosis may often require the application of multiple different active agents to a biological interface. For example, when performing allergy testing, a patient will receive numerous injections, each delivering a separate allergen to a respective portion of the biological interface. For example, a patient may receive from six (6) to twelve (12) separate injections in a visit. Each allergen is spatially distributed on the biological interface. After a period of time, the medical service provider will check for reaction at each location. Another series of multiple injections may follow, whether or not a reaction from the previous series is detected. Such an approach is time consuming for both the patient and the medical service provider. Such an approach is also tedious, and quite painful for the patient. Additionally, such an approach generates an excessive amount of medical waste (e.g., spent syringes and needles, and spent containers of allergen), which requires special handling and costly disposal. An improved approach that addresses at least some of the problems is desirable.

### BRIEF SUMMARY OF THE INVENTION

According to one embodiment, an iontophoresis device operable to deliver active agents to a biological interface of a biological entity, comprises: an active electrode assembly, the active electrode assembly including a contact face exposed on an exterior of the active electrode to be proximate to a biological interface when in use, an active electrode element operable to apply a first electrical potential, a first active agent reservoir capable of storing a first active agent, at least a second active agent reservoir capable of storing a second active agent, an outermost ion selective membrane exposed to the exterior of the iontophoresis device to form an interface with the biological interface, the outermost ion selective membrane substantially permeable by ions having a first polarity that matches a polarity of the first and the second active agents, and substantially impermeable by ions of a second polarity, opposite the first polarity, at least a portion of the first and second active agent reservoirs formed in the outermost ion selective membrane, the second active agent reservoir spaced laterally in a plane approximately parallel to the contact face from the first active agent reservoir, at least the first and the second active agent reservoirs positioned with respect to the active electrode element to each actively transfer at least some of the first and the second active agents from the iontophoresis device to the biological interface in response to application of the first electrical potential; and a counter electrode assembly spaced laterally from the active electrode assembly, the counter electrode assembly including a counter electrode element operable to apply a second electrical potential, the second electrical potential being different from the first electrical potential.

According to one embodiment, an active agent delivery system operable to deliver active agents to at least two distinct areas on a biological interface, comprises: an active electrode element operable to provide a first electrical potential; and a retaining structure having at least two receptacles, each of the receptacles configured to securely receive a respective active agent reservoir, the receptacles spaced laterally with respect to each other to overlie respective ones of the distinct areas on the biological surface when the active agent delivery system is in use; each of the receptacles at least partially underlying the active electrode element.

According to one embodiment, an active agent delivery system, comprises: an active electrode element operable to provide an electromotive force or current; an outer ion selective membrane having an outer surface and at least two distinct regions laterally spaced from one another across the outer surface, each of the distinct regions having pores; and at least two active agents of a first polarity cached within the pores of respective ones of the distinct regions of the ion selective membrane and substantially retained therein in the absence of the electromotive force or current and transferred outwardly from the ion selective membrane in the presence of the electromotive force or current.

### BRIEF DESCRIPTION OF THE SEVERAL VIEWS OF THE DRAWING(S)

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn are not intended to convey any information regarding the actual shape of the particular elements and have been solely selected for ease of recognition in the drawings.

FIG. 1 is a block diagram of an iontophoresis device comprising active and counter electrode assemblies according to one illustrated embodiment where the active electrode assembly includes a retaining structure, multiple active agent reservoirs, an outermost membrane caching an active agent, active agent adhered to an outer surface of the outermost membrane and a removable outer release liner overlying or covering the active agent and outermost membrane.

FIG. 2 is a block diagram of the iontophoresis device of FIG. 1 positioned on a biological interface, with the outer release liner removed to expose the active agent according to one illustrated embodiment.

FIG. 3 is an isometric view of the retaining structure of FIG. 1, showing the multiple active agent reservoirs with one active agent reservoir positioned for insertion into a receptacle of the retaining structure.

FIG. 4 is a block diagram of an iontophoresis device comprising active and counter electrode assemblies according to another illustrated embodiment where the active electrode assembly includes a retaining structure having at least two laterally spaced receptacles, at least two active agent reservoirs insertably secured within the laterally spaced receptacles, and a blister pack having blisters of hydrating agent and/or active agent.

FIG. 5 is a partially exploded block diagram of an active electrode assembly of an iontophoresis device, showing a retaining structure having at least two laterally spaced receptacles, at least two active agent reservoirs, and a blister pack, with one of the active agent reservoirs positioned for insertion and the blister pack positioned to contact an outer surface of the active agent reservoirs.

FIG. 6 is a bottom plan view of a retaining structure in an active electrode assembly, showing at least two active agent reservoirs.

FIG. 7 is a top plan view of a blister pack, showing at least two blisters and an aligning mechanism.

### DETAILED DESCRIPTION OF THE INVENTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with iontophoresis devices including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “in another embodiment” means that a particular referent feature, structure, or characteristic described in connection with the embodiment is included in at least one embodiment. Thus, the appearance of the phrases “in one embodiment,” or “in an embodiment,” or “in another embodiment” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to an iontophoresis device including “an electrode element” includes a single electrode element, or two or more electrode elements. It should also be noted that the term “or” is generally employed in its sense including “and/or” unless the content clearly dictates otherwise.

As used herein the term “membrane” means a boundary, a layer, barrier, or material, which may, or may not be permeable. The term “membrane” may further refer to an interface. Unless specified otherwise, membranes may take the form of a solid, liquid, or gel, and may or may not have a distinct lattice, non cross-linked structure, or cross-linked structure.

As used herein the term “ion selective membrane” means a membrane that is substantially selective to ions, passing certain ions while blocking passage of other ions. An ion selective membrane, for example, may take the form of a charge selective membrane, or may take the form of a semi-permeable membrane.

As used herein the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH, and ACS also from Tokuyama Co., Ltd.

As used herein and in the claims, the term “bipolar membrane” means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion opposed to the first portion, including anion ion exchange materials or groups. The multiple membrane structure (e.g., two film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules at a first rate, and some other molecules at a second rate different than the first. In yet further embodiments, the “semi-permeable membrane” may take the form of a selectively permeable membrane allowing only certain selective molecules to pass through it.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “gel matrix” means a type of reservoir, which takes the form of a three dimensional network, a colloidal suspension of a liquid in a solid, a semi-solid, a cross-linked gel, a non cross-linked gel, a jelly-like state, and the like. In some embodiments, the gel matrix may result from a three dimensional network of entangled macromolecules (e.g., cylindrical micelles). In some embodiments, a gel matrix may include hydrogels, organogels, and the like. Hydrogels refer to three-dimensional network of, for example, cross-linked hydrophilic polymers in the form of a gel and substantially composed of water. Hydrogels may have a net positive or negative charge, or may be neutral.

As used herein and in the claims, the term “reservoir” means any form of mechanism to retain an element, compound, pharmaceutical composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and/or current into the biological interface. A reservoir may also retain an electrolyte solution.

As used herein and in the claims, the term “active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., a drug, a therapeutic compound, pharmaceutical salts, and the like) non-pharmaceuticals (e.g., cosmetic substance, and the like), a vaccine, an immunological agent, a local or general anesthetic or painkiller, an antigen or a protein or peptide such as insulin, a chemotherapy agent, an anti-tumor agent.

In some embodiments, the term “active agent” further refers to the active agent, as well as its pharmacologically active salts, pharmaceutically acceptable salts, prodrugs, metabolites, analogs, and the like. In some further embodiment, the active agent includes at least one ionic, cationic, ionizeable, and/or neutral therapeutic drug and/or pharmaceutical acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. Other active agents may be polarized or polarizable, that is exhibiting a polarity at one portion relative to another portion. For instance, an active agent having an amino group can typically take the form an ammonium salt in solid state and dissociates into a free ammonium ion (NH₄⁺) in an aqueous medium of appropriate pH.

The term “active agent” may also refer to neutral agents, molecules, or compounds capable of being delivered via electro-osmotic flow. The neutral agents are typically carried by the flow of, for example, a solvent during electrophoresis. Selection of the suitable active agents is therefore within the knowledge of one skilled in the relevant art.

In some embodiments, one or more active agents may be selected from analgesics, anesthetics, anesthetics vaccines, antibiotics, adjuvants, immunological adjuvants, immunogens, tolerogens, allergens, toll-like receptor agonists, toll-like receptor antagonists, immuno-adjuvants, immuno-modulators, immuno-response agents, immuno-stimulators, specific immuno-stimulators, non-specific immuno-stimulators, and immuno-suppressants, or combinations thereof.

Non-limiting examples of such active agents include lidocaine, articaine, and others of the -caine class; morphine, hydromorphone, fentanyl, oxycodone, hydrocodone, buprenorphine, methadone, and similar opioid agonists; sumatriptan succinate, zolmitriptan, naratriptan HCl, rizatriptan benzoate, almotriptan malate, frovatriptan succinate and other 5-hydroxytryptaminel receptor subtype agonists; resiquimod, imiquidmod, and similar TLR 7 and 8 agonists and antagonists; domperidone, granisetron hydrochloride, ondansetron and such anti-emetic drugs; zolpidem tartrate and similar sleep inducing agents; L-dopa and other anti-Parkinson's medications; aripiprazole, olanzapine, quetiapine, risperidone, clozapine, and ziprasidone, as well as other neuroleptica; diabetes drugs such as exenatide; as well as peptides and proteins for treatment of obesity and other maladies.

Further non-limiting examples of anesthetic active agents or pain killers include ambucaine, amethocaine, isobutyl p-aminobenzoate, amolanone, amoxecaine, amylocaine, aptocaine, azacaine, bencaine, benoxinate, benzocaine, N,N-dimethylalanylbenzocaine, N,N-dimethylglycylbenzocaine, glycylbenzocaine, beta-adrenoceptor antagonists betoxycaine, bumecaine, bupivicaine, levobupivicaine, butacaine, butamben, butanilicaine, butethamine, butoxycaine, metabutoxycaine, carbizocaine, carticaine, centbucridine, cepacaine, cetacaine, chloroprocaine, cocaethylene, cocaine, pseudococaine, cyclomethycaine, dibucaine, dimethisoquin, dimethocaine, diperodon, dyclonine, ecognine, ecogonidine, ethyl aminobenzoate, etidocaine, euprocin, fenalcomine, fomocaine, heptacaine, hexacaine, hexocaine, hexylcaine, ketocaine, leucinocaine, levoxadrol, lignocaine, lotucaine, marcaine, mepivacaine, metacaine, methyl chloride, myrtecaine, naepaine, octacaine, orthocaine, oxethazaine, parenthoxycaine, pentacaine, phenacine, phenol, piperocaine, piridocaine, polidocanol, polycaine, prilocaine, pramoxine, procaine (Novocaine®), hydroxyprocaine, propanocaine, proparacaine, propipocaine, propoxycaine, pyrrocaine, quatacaine, rhinocaine, risocaine, rodocaine, ropivacaine, salicyl alcohol, tetracaine, hydroxytetracaine, tolycaine, trapencaine, tricaine, trimecaine tropacocaine, zolamine, a pharmaceutically acceptable salt thereof, and mixtures thereof.

As used herein and in the claims, the term “subject” generally refers to any host, animal, vertebrate, or invertebrate, and includes fish, mammals, amphibians, reptiles, birds, and particularly humans.

As used herein and in the claims, the term “agonist” refers to a compound that can combine with a receptor (e.g., a Toll-like receptor, and the like) to produce a cellular response. An agonist may be a ligand that directly binds to the receptor. Alternatively, an agonist may combine with a receptor indirectly by forming a complex with another molecule that directly binds the receptor, or otherwise resulting in the modification of a compound so that it directly binds to the receptor.

As used herein and in the claims, the term “antagonist” refers to a compound that can combine with a receptor (e.g., a Toll-like receptor, and the like) to inhibit a cellular response. An antagonist may be a ligand that directly binds to the receptor. Alternatively, an antagonist may combine with a receptor indirectly by forming a complex with another molecule that directly binds to the receptor, or otherwise results in the modification of a compound so that it directly binds to the receptor.

As used herein and in the claims, the term “effective amount” or “therapeutically effective amount” includes an amount effective at dosages and for periods of time necessary, to achieve the desired result. The effective amount of a composition containing a pharmaceutical agent may vary according to factors such as the disease state, age, gender, and weight of the subject.

As used herein and in the claims, the term “analgesic” refers to an agent that lessens, alleviates, reduces, relieves, or extinguishes a neural sensation in an area of a subject's body. In some embodiments, the neural sensation relates to pain, in other aspects the neural sensation relates to discomfort, itching, burning, irritation, tingling, “crawling,” tension, temperature fluctuations (such as fever), inflammation, aching, or other neural sensations.

As used herein and in the claims, the term “anesthetic” refers to an agent that produces a reversible loss of sensation in an area of a subject's body. In some embodiments, the anesthetic is considered to be a “local anesthetic” in that it produces a loss of sensation only in one particular area of a subject's body.

As used herein and in the claims, the term “allergen” refers to any agent that elicits an allergic response. Some examples of allergens include but are not limited to chemicals and plants, drugs (such as antibiotics, serums), foods (such as milk, wheat, eggs, etc), bacteria, viruses, other parasites, inhalants (dust, pollen, perfume, smoke), and/or physical agents (heat, light, friction, radiation). As used herein, an allergen may be an immunogen.

As used herein and in the claims, the term “adjuvant” and any derivations thereof, refers to an agent that modifies the effect of another agent while having few, if any, direct effect when given by itself. For example, an adjuvant may increase the potency or efficacy of a pharmaceutical, or an adjuvant may alter or affect an immune response.

As used herein and in the claims, the terms “vehicle,” “carrier,” “pharmaceutically vehicle,” “pharmaceutically carrier,” “pharmaceutically acceptable vehicle,” or “pharmaceutically acceptable carrier” may be used interchangeably, and refer to pharmaceutically acceptable solid or liquid, diluting or encapsulating, filling or carrying agents, which are usually employed in pharmaceutical industry for making pharmaceutical compositions. Examples of vehicles include any liquid, gel, salve, cream, solvent, diluent, fluid ointment base, vesicle, liposomes, nisomes, ethasomes, transfersomes, virosomes, cyclic oligosaccharides, non ionic surfactant vesicles, phospholipid surfactant vesicles, micelle, and the like, that is suitable for use in contacting a subject.

In some embodiments, the pharmaceutical vehicle may refer to a composition that includes and/or delivers a pharmacologically active agent, but is generally considered to be otherwise pharmacologically inactive. In some other embodiments, the pharmaceutical vehicle may have some therapeutic effect when applied to a site such as a mucous membrane or skin, by providing, for example, protection to the site of application from conditions such as injury, further injury, or exposure to elements. Accordingly, in some embodiments, the pharmaceutical vehicle may be used for protection without a pharmacological agent in the formulation.

The headings provided herein are for convenience only and do not interpret the scope or meaning of the embodiments.

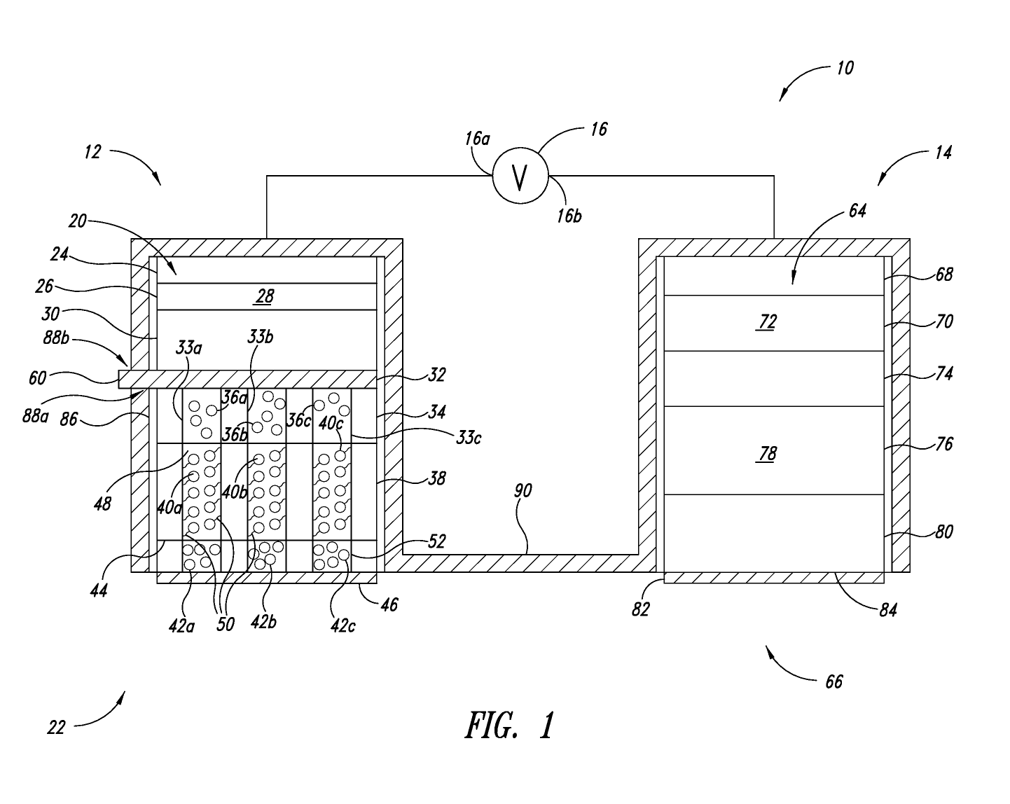

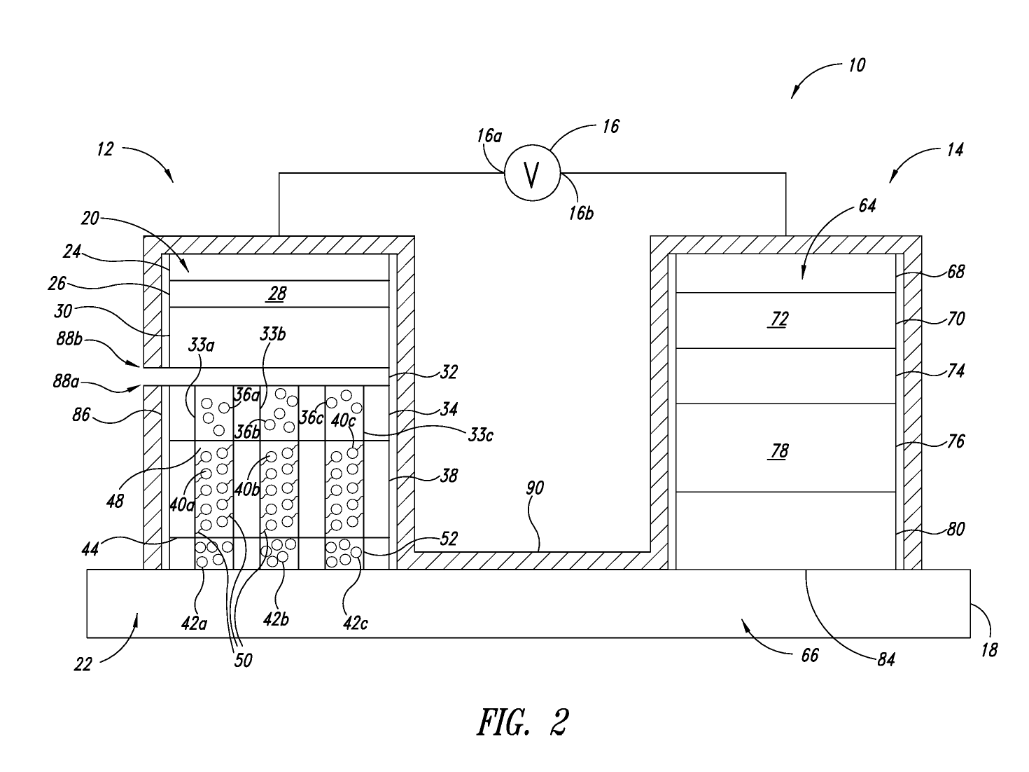

FIGS. 1 and 2 show an iontophoresis device 10 comprising active and counter electrode assemblies, 12, 14, respectively, electrically coupled to a voltage source 16, operable to supply an active agent to a biological interface 18 (FIG. 2), such as a portion of skin or mucous membrane via iontophoresis, according to one illustrated embodiment.

In simpler embodiments (not shown), the active electrode assembly 12 may include an active electrode element 24, at least two laterally spaced active agent reservoirs 33a-33c (collectively 33), and at least two active agents 36a-36c (collectively 36). In the illustrated embodiment, the active electrode assembly 12 comprises, from an interior 20 to an exterior 22 of the active electrode assembly 12, an active electrode element 24, an electrolyte reservoir 26 storing an electrolyte 28, an inner ion selective membrane 30, an inner sealing liner 32, at least two laterally spaced active agent reservoirs 33a-33c storing active agents 36a-36c, a retaining structure 34 having at least two laterally spaced receptacles to retain respective ones of the active agent reservoirs 33a-33c, an outermost ion selective membrane 38 that optionally caches additional active agents 40a-40c (collectively 40), optional, further active agents 42a-42c (collectively 42) carried by an outer surface 44 of the outermost ion selective membrane 38, and an outer release liner 46. Each of the above elements or structures will be discussed in detail below.

The active electrode element 24 is coupled to a first pole 16a of the voltage source 16 and positioned in the active electrode assembly 12 to apply an electromotive force or current to transport active agents 36, 40, 42, via various other components of the active electrode assembly 12. The active electrode element 24 may take a variety of forms. For example, the active electrode element 24 may include a sacrificial element, for example a chemical compound or amalgam including silver (Ag) or silver chloride (AgCl). Such compounds or amalgams typically employ one or more heavy metals, for example lead (Pb), which may present issues with regard to manufacturing, storage, use and/or disposal. Consequently, some embodiments may advantageously employ a carbon-based active electrode element 24. Such may, for example, comprise multiple layers, for example a gel or polymer matrix comprising carbon and a conductive sheet comprising carbon fiber or carbon fiber paper, such as that described in commonly assigned pending Japanese patent application 2004/317317, filed Oct. 29, 2004.

The electrolyte reservoir 26 may take a variety of forms including any structure capable of retaining electrolyte 28, and in some embodiments may even be the electrolyte 28 itself, for example, where the electrolyte 28 is in a gel, semi-solid or solid form. For example, the electrolyte reservoir 26 may take the form of a pouch or other receptacle, a membrane with pores, cavities or interstices, particularly where the electrolyte 28 is a liquid.

The electrolyte 28 may provide ions or donate charges to prevent or inhibit the formation of gas bubbles (e.g., hydrogen) on the active electrode element 24 in order to enhance efficiency and/or increase delivery rates. This elimination or reduction in electrolysis may in turn inhibit or reduce the formation of acids and/or bases (e.g., H⁺ ions, OH⁻ ions), that would otherwise present possible disadvantages such as reduced efficiency, reduced transfer rate, and/or possible irritation of the biological interface 18. As discussed further below, in some embodiments the electrolyte 28 may provide or donate ions to substitute for the active agent 40 cached in the outermost ion selective membrane 38. Such may facilitate transfer of the active agent 40 to the biological interface 18, for example, increasing and/or stabilizing delivery rates. A suitable electrolyte may take the form of a solution of 0.5M disodium fumarate: 0.5M Poly acrylic acid (5:1).

The inner ion selective membrane 30 is generally positioned to separate the electrolyte 28 and the active agent reservoirs 33. The inner ion selective membrane 30 may take the form of a charge selective membrane. For example, where the active agents 36, 40, 42 comprise a cationic active agent, the inner ion selective membrane 38 may take the form of an anion exchange membrane, selective to substantially pass anions and substantially block cations. Also, for example, where the active agent 36, 40, 42 comprise an anionic active agent, the inner ion selective membrane 38 may take the form of a cationic exchange membrane, selective to substantially pass cations and substantially block anions. The inner ion selective membrane 38 may advantageously prevent transfer of undesirable elements or compounds between the electrolyte 28 and the active agent 36, 40, 42. For example, the inner ion selective membrane 38 may prevent or inhibit the transfer of hydrogen (H⁺) or sodium (Na⁺) ions from the electrolyte 72, which may increase the transfer rate and/or biological compatibility of the iontophoresis device 10.

The inner sealing liner 32 is optional, and separates the active agents 36, 40, 42 from the electrolyte 28 and is selectively removable. The inner sealing liner 32 may advantageously prevent migration or diffusion between the active agents 36, 40, 42 and the electrolyte 28, for example, during storage.

The active agent reservoirs 33 are generally positioned between the inner ion selective membrane 30 and the outermost ion selective membrane 38, and can be secured in retaining structure 34. The retaining structure 34 can receive and retain active agent reservoirs 33, and can be any structure with laterally spaced cavities, pores, receptacles, or any void or formation that can maintain the active agent reservoirs 33a-33c spatially separated laterally. Active agent reservoirs 33 may take a variety of forms including any structure capable of temporarily retaining active agents 36, and in some embodiments may even be the active agents 36a-36c itself, for example, where the active agent is in a gel, semi-solid or solid form. For example, the active agent reservoirs 33 may take the form of a pouch or other receptacle, a membrane with pores, cavities or interstices, particularly where the active agent 36 is a liquid. The active agent reservoirs 33 may advantageously allow larger doses of the active agent 36 to be loaded in the active electrode assembly 12.

Two or more of the active agents 36a-36c may each be the same composition in some embodiments, or in other embodiments, they may each be distinct compounds or elements. For example, in at least one embodiment, the active agents 36a-36c, 40a-40c, 42a-42c, may comprise multiple antigens for allergy screening tests, where all antigens may be administered simultaneously, eliminating the need for the antigens to be individually injected. In another embodiment, each of the active agent reservoirs 33a-33c may store a respective active agent 36a-36c that are either inconvenient or inefficient to consume orally or by injection, or must be delivered on a repetitive basis. In such embodiments the active agents can be delivered simultaneously without administration by oral means or injection.

The outermost ion selective membrane 38 is positioned generally opposed across the active electrode assembly 12 from the active electrode element 24. The outermost membrane 38 may, as in the embodiment illustrated in FIGS. 1 and 2, take the form of an ion exchange membrane, pores 48 (only one called out in FIGS. 1 and 2 for sake of clarity of illustration) of the ion selective membrane 38 including ion exchange material or groups 50 (only three called out in FIGS. 1 and 2 for sake of clarity of illustration). Under the influence of an electromotive force or current, the ion exchange material or groups 50 selectively substantially passes ions of the same polarity as active agents 36, 40, 42 while substantially blocking ions of the opposite polarity. Thus, the outermost ion exchange membrane 38 is charge selective. Where the active agent 36, 40, 42 is a cation (e.g., lidocaine), the outermost ion selective membrane 38 may take the form of a cation exchange membrane. Alternatively, where the active agent 36, 40, 42 is an anion, the outermost ion selective membrane 38 may take the form of an anion exchange membrane.

The outermost ion selective membrane 38 may advantageously cache at least two active agents 40a-40c. In particular, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

Alternatively, the outermost ion selective membrane 38 may take the form of a semi-permeable or microporous membrane which is selective by size. In some embodiments, such a semi-permeable membrane may advantageously cache active agents 40a-40c, for example by employing the removably releasable outer release liner 46 to retain the active agents 40a-40c, until the outer release liner 46 is removed prior to use. Another embodiment (not shown) may exclude the outermost ion selective membrane 38 and may employ the removably releasable outer release liner 46 to retain the active agents 36a-36c, stored in active agent reservoirs 33a-33c, respectively, until the outer release liner 46 is removed prior to use.

The outermost ion selective membrane 38 may be preloaded with the additional active agents 40a-40c, such as ionized or ionizable drugs or therapeutic agents and/or polarized or polarizable drugs or therapeutic agents. Where the outermost ion selective membrane 38 is an ion exchange membrane, a substantial amount of active agents 40 may bond to ion exchange groups 50 in the pores, cavities or interstices 48 of the outermost ion selective membrane 38. In at least one embodiment (not shown), the outer most ion selective membrane 38 may itself be a retaining structure and the pores 48 may serve as active agent reservoirs, eliminating the need for a distinct retaining structure 34 and active agent reservoirs 33.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically, vapor deposition, and/or otherwise. In some embodiments, the further active agent 42a-42c may sufficiently cover respective portions of the outer surface 44 and/or be of sufficient thickness so as to form distinct layers 52 (only one called out in FIGS. 1 and 2 for sake of clarity of illustration). In other embodiments, the further active agent 42 may not be sufficient in volume, thickness or coverage as to constitute a layer in a conventional sense of such term.

The active agent 42 may be deposited in a variety of highly concentrated forms such as, for example, solid form, nearly saturated solution form or gel form. If in solid form, a source of hydration may be provided, either integrated into the active electrode assembly 12, or applied from the exterior thereof just prior to use.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. Thus, a first set of distinct types of active agents may be stored in the inner active agent reservoirs 33, while a second distinct set of types of active agents may be cached in the outermost ion selective membrane 38. In such an embodiment, either the first set or the second set of active agents or a combination thereof may be deposited on the outer surface 44 of the outermost ion selective membrane 38 as the further active agent 42. Alternatively, a mix of the first and the second sets of active agents may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first set of active agents may be stored in the inner active agent reservoirs 33 as the active agents 36, and cached in the outermost ion selective membrane 38 as the additional active agents 40, while a second type of active agent may be deposited on the outer surface 44 of the outermost ion selective membrane 38 as the further active agent 42. Typically, in embodiments where one or more different active agents are positioned in the device 10 in a longitudinal rather than lateral fashion. The active agents 36, 40, 42 will typically be of common polarity to prevent the active agents 36, 40, 42 from competing with one another. Other combinations are possible.

The spacing of active agents 36, 40, 42 longitudinally will typically lend to a temporal separation in delivery of the respective active agent, the further active agent 42 being delivered first, the additional active agent 40 being delivered second, and the active agent 36 being delivered last. This contrasts with the lateral spacing of the active agents across a face of the active electrode assembly 12. Such a distribution will generally first deliver the active agents 42a-42c substantially simultaneously, barring significant differences in the transfer numbers of the particular active agents 42a-42c. Then the additional active agents 40a-40c will be delivered all at approximately the same time as one another, again barring significant differences in their transfer numbers. Finally, the active agents 36a-36c will all be delivered at approximately the same time as one another, barring significant differences in their transfer numbers.

The outer release liner 46 may generally be positioned overlying or covering further active agents 42 carried by the outer surface 44 of the outermost ion selective membrane 38. The outer release liner 46 may protect the further active agents 42 and/or outermost ion selective membrane 38 during storage, prior to application of an electromotive force or current. The outer release liner 46 may be a selectively releasable liner made of waterproof material, such as release liners commonly associated with pressure sensitive adhesives. Note that the inner release liner 46 is shown in place in FIG. 1 and removed in FIG. 2. It is also possible in other embodiments (not shown) that the outer surface 44 is contiguous to the outer release liner 46, precluding a layer of further active agents 42 from forming. In such embodiments, the outer release liner 46 may protect the outermost ion selective membrane 38. In other embodiments where the outermost ion selective membrane is eliminated, the outer release liner 46 may protect reservoirs 33 and active agents 36.

An interface coupling medium (not shown) may be employed between the electrode assembly and the biological interface 18. The interface coupling medium may, for example, take the form of an adhesive and/or gel. The gel may, for example, take the form of a hydrating gel.

The counter electrode assembly 14 allows completion of an electrical path between poles 16a, 16b of the voltage source 16 via the active electrode assembly 12 and the biological interface 18. The counter electrode assembly 14 may take a variety of forms suitable for closing the circuit by providing a return path.

In the embodiment illustrated in FIGS. 1 and 2, the counter electrode assembly 14 comprises, in order from an interior 64 to an exterior 66 of the counter electrode assembly 14: a counter electrode element 68, electrolyte reservoir 70 storing an electrolyte 72, an inner ion selective membrane 74, an optional buffer reservoir 76 storing buffer material 78, an outermost ion selective membrane 80, and an outer release liner 82 (FIG. 1).

The counter electrode element 68 is electrically coupled to a second pole 16b of the voltage source 16, the second pole 16b having an opposite polarity to the first pole 16a. The counter electrode element 68 may take a variety of forms. For example, the counter electrode element 68 may include a sacrificial element, such as a chemical compound or amalgam including silver (Ag) or silver chloride (AgCl), or may include a non-sacrificial element such as the carbon-based electrode element discussed above.

The electrolyte reservoir 70 may take a variety of forms including any structure capable of retaining electrolyte 72, and in some embodiments may even be the electrolyte 72 itself, for example, where the electrolyte 72 is in a gel, semi-solid or solid form. For example, the electrolyte reservoir 70 may take the form of a pouch or other receptacle, or a membrane with pores, cavities or interstices, particularly where the electrolyte 72 is a liquid.

The electrolyte 72 is generally positioned between the counter electrode element 68 and the outermost ion selective membrane 80, proximate to the counter electrode element 68. The electrolyte 72 may provide ions or donate charges to prevent or inhibit the formation of gas bubbles (e.g., hydrogen) on the counter electrode element 68 and may prevent or inhibit the formation of acids or bases or neutralize the same, which may enhance efficiency and/or reduce the potential for irritation of the biological interface 18 (FIG. 2).

The inner ion selective membrane 74 is positioned between and/or to separate, the electrolyte 72 from the buffer material 78. The inner ion selective membrane 74 may take the form of a charge selective membrane, such as the illustrated ion exchange membrane that substantially allows passage of ions of a first polarity or charge while substantially blocking passage of ions or charge of a second, opposite polarity. The inner ion selective membrane 74 will typically pass ions of opposite polarity or charge to those passed by the outermost ion selective membrane 80 while substantially blocking ions of like polarity or charge. Alternatively, the inner ion selective membrane 74 may take the form of a semi-permeable or microporous membrane that is selective based on size.

The inner ion selective membrane 74 may prevent transfer of undesirable elements or compounds into the buffer material 78. For example, the inner ion selective membrane 74 may prevent or inhibit the transfer of hydrogen (H⁺) or sodium (Na⁺) ions from the electrolyte 72 into the buffer material 78.

The optional buffer reservoir 76 is generally disposed between the electrolyte reservoir 70 and the outermost ion selective membrane 80. The buffer reservoir 76 may take a variety of forms capable of temporarily retaining the buffer material 78. For example, the buffer reservoir 76 may take the form of a cavity, a porous membrane or a gel.

The buffer material 78 may supply ions for transfer through the outermost ion selective membrane 80 to the biological interface 18. Consequently, the buffer material 78 may, for example, comprise a salt (e.g., NaCl).

The outermost ion selective membrane 80 of the counter electrode assembly 14 may take a variety of forms. For example, the outermost ion selective membrane 80 may take the form of a charge selective ion exchange membrane, such as a cation exchange membrane or an anion exchange membrane, which substantially passes and/or blocks ions based on the charge carried by the ion. Examples of suitable ion exchange membranes are discussed above. Alternatively, the outermost ion selective membrane 80 may take the form of a semi-permeable membrane that substantially passes and/or blocks ions based on size or molecular weight of the ion.

The outermost ion selective membrane 80 of the counter electrode assembly 14 is selective to ions with a charge or polarity opposite to that of the outermost ion selective membrane 38 of the active electrode assembly 12. Thus, for example, where the outermost ion selective membrane 38 of the active electrode assembly 12 allows passage of negatively charged ions of the active agents 36, 40, 42 to the biological interface 18, the outermost ion selective membrane 80 of the counter electrode assembly 14 allows passage of positively charged ions to the biological interface 18, while substantially blocking passage of ions having a negative charge or polarity. On the other hand, where the outermost ion selective membrane 38 of the active electrode assembly 12 allows passage of positively charged ions of the active agents 36, 40, 42 to the biological interface 18, the outermost ion selective membrane 80 of the counter electrode assembly 14 allows passage of negatively charged ions to the biological interface 18 while substantially blocking passage of ions with a positive charge or polarity.

The outer release liner 82 (FIG. 1) may generally be positioned overlying or covering an outer surface 84 of the outermost ion selective membrane 80. Note that the inner release liner 82 is shown in place in FIG. 1 and removed in FIG. 2. The outer release liner 82 may protect the outermost ion selective membrane 80 during storage, prior to application of an electromotive force or current. The outer release liner 82 may be a selectively releasable liner made of waterproof material, such as release liners commonly associated with pressure sensitive adhesives. In some embodiments, the outer release liner 82 may be coextensive with the outer release liner 46 of the active electrode assembly 12.

The voltage source 16 may take the form of one or more chemical battery cells, super- or ultra-capacitors, or fuel cells. The voltage source 16 may be selectively electrically coupled to the active and counter electrode assemblies 12, 14 via a control circuit (not shown), which may include discrete and/or integrated circuit elements to control the voltage, current and/or power delivered to the electrode assemblies 12, 14.

As suggested above, the active agents 36, 40, 42 may take the form of a cationic or an anionic drug or other therapeutic agent. Consequently, the terminals or poles 16a, 16b of the voltage source 16 may be reversed. Likewise, the selectivity of the outermost ion selective membranes 38, 80 and inner ion selective membranes 30, 74 may be reversed.

The iontophoresis device 10 may further comprise an inert molding material 86 adjacent exposed sides of the various other structures forming the active and counter electrode assemblies 12, 14. The molding material 86 may advantageously provide environmental protection to the various structures of the active and counter electrode assemblies 12, 14. Molding material 86 may form a slot or opening 88a on one of the exposed sides through which the tab 60 (FIG. 1) extends to allow for the removal of inner sealing liner 32 prior to use. Enveloping the active and counter electrode assemblies 12, 14 is a housing material 90. The housing material 90 may also form a slot or opening 88b positioned aligned with the slot or opening 88a in molding material 86 through which the tab 60 extends to allow for the removal of inner sealing liner 32 prior to use of the iontophoresis device 10, as described below.

Immediately prior to use, the iontophoresis device 10 is prepared by withdrawing the inner sealing liner 32 and removing the outer release liners 46, 82. As described above, the inner sealing liner 32 may be withdrawn by pulling on tab 60. The outer release liners 46, 82 may be pulled off in a similar fashion to removing release liners from pressure sensitive labels and the like.

As best seen in FIG. 2, the active and counter electrode assemblies 12, 14 are positioned on the biological interface 18. Positioning on the biological interface 18 may close the circuit, allowing electromotive force to be applied and/or current to flow from one pole 16a of the voltage source 16 to the other pole 16b, via the active electrode assembly, biological interface 18 and counter electrode assembly 14.

In the presence of the electromotive force and/or current, active agents 36 are transported toward the biological interface 18. Additional active agents 40 are released by the ion exchange groups or material 50 by the substitution of ions of the same charge or polarity (e.g., active agent 36a-36c), and transported toward the biological interface 18. While some of the active agents 36 may substitute for the additional active agents 40 some of the active agents 36 may be transferred through the outermost ion elective membrane 38 into the biological interface 18. Further active agents 42, if any, carried by the outer surface 44 of the outermost ion elective membrane 38, are also transferred to the biological interface 18.

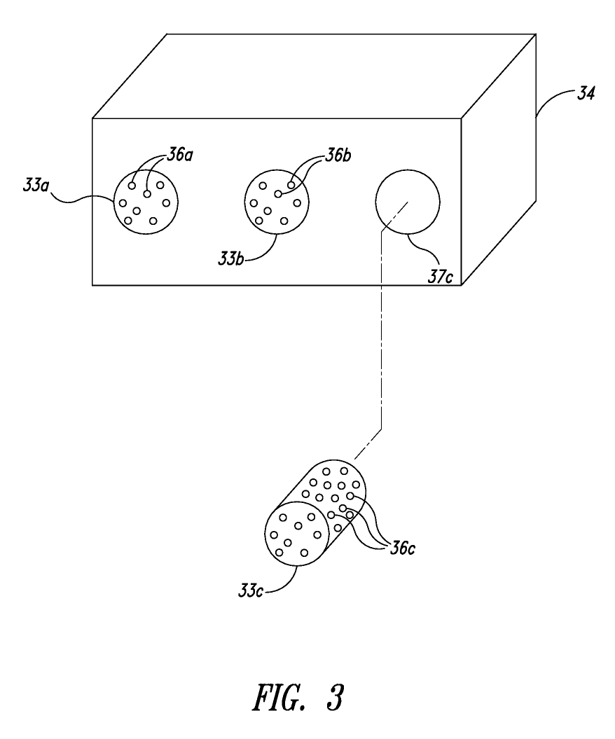

FIG. 3 shows one exemplary embodiment of the retaining structure 34 with one of the active agent reservoirs 33c awaiting insertion into a receptacle 37c. Retaining structure 34 can receive and retain at least two active agent reservoirs 33a-33c, allowing the active agent reservoirs 33a-33c to store substantially the same or substantially distinct active agents 36a-36c. In one illustrated embodiment as shown in FIG. 3, retaining structure 34 retains three active agent reservoirs 33a-33c laterally spaced across a plane that is approximately parallel to a contact face of the active electrode assembly 12 (FIGS. 1).

Retaining structure 34 can be fixedly positioned in the iontophoretic device 10. Alternatively, or additionally, retaining structure 34 may be in cartridge form removably secured in the iontophoretic device 10. In cartridge form, the retaining structure 34 can be removed and replaced when active agents 36 are depleted or after use on a first patient, to ready the device for a next patient. Such may advantageously allow patient contacting portions to be removed and disposed of for sanitary purposes. Such may also permit the removal of portions that would not be capable of undergoing sterilization procedures such as exposure to high temperatures or strong chemicals (e.g., bleach).

In such embodiments, multiple retaining structure 34 cartridges can be utilized with one iontophoretic device 10, adding to the commercial viability of the device. In some embodiments, the active agent reservoirs 33 can be insertably retained in retaining structure 34, whereas, in other embodiments, the active agent reservoirs 33 are formed in the retaining structure 34 as cavities, pores, receptacles, and/or any other void capable of storing active agent. In still other embodiments active agents 36a-36c can be injected into active agent reservoirs 33 via a syringe or other device for one time use or to refill the active agent reservoirs 33 for reuse.

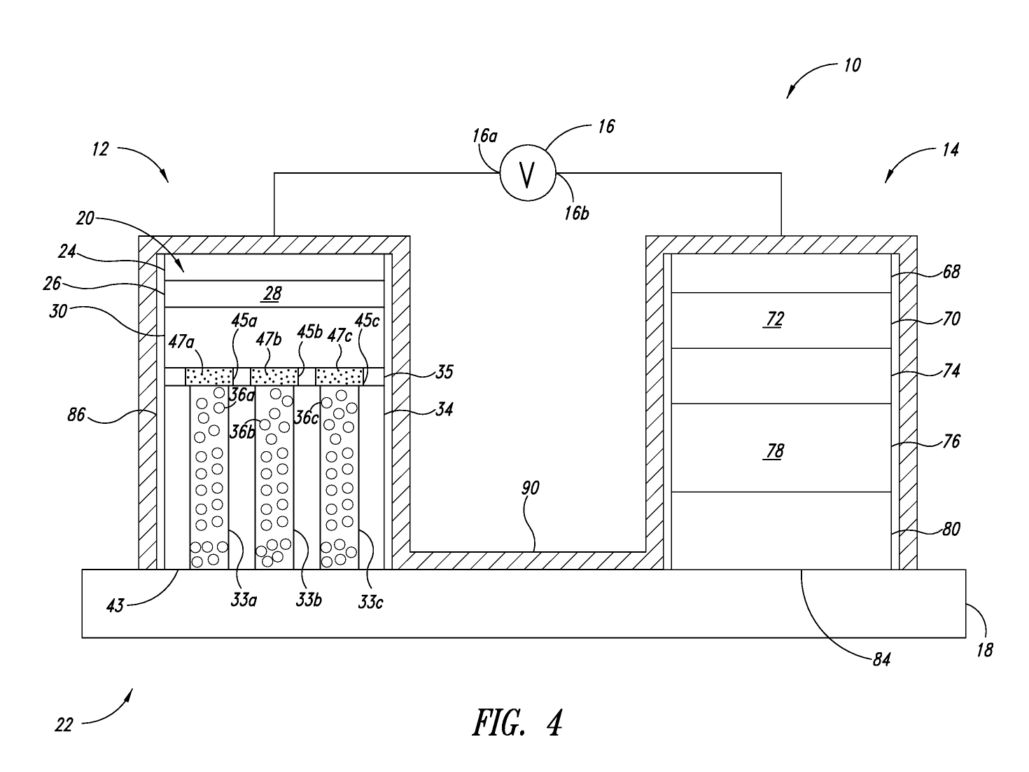

FIG. 4 shows another embodiment of the iontophoretic device 10, including a blister pack 35 situated adjacent or at least proximate to the retaining structure 34, which receives active agent reservoirs 33. As shown in FIG. 4, at least two active agent reservoirs 33a-33c are spatially separated laterally from one another in a plane that is approximately parallel to a contact face 43 of the active electrode assembly 12. The blister pack 35 may comprise distinct blisters 45a-45c (collectively 45), storing hydrating agents 47a-47c (collectively 47). Blisters 45 can be positioned adjacent or at least proximate to the active agent reservoirs 33. In such embodiments, active agents 36 can be in dehydrated form prior to use. Selectively pressing and breaking the blisters 45 hydrates selected active agents 36a-36c at the time of use so that through the electromotive force across the electrode assemblies 12 and 14, as described, charged active agent molecules, as well as ions and other charged components, transfer through the biological interface 18 into the biological tissue. Such embodiments can be advantageous for applications requiring repetitive active agent doses at certain time intervals. For example, different doses or different active agents 36a-36c may be stored in active agent reservoirs 33, each corresponding to a blister 45a-45c in blister pack 35. The blisters 45 can be separately and/or individually pressed and broken at prescribed active agent administration intervals. The use of a blister pack 35 can also prevent errors in over-transfer or under-transfer of active agent since it will be clear from the appearance of the blisters 45 how many doses or which active agents have been previously migrated through the biological interface 18.

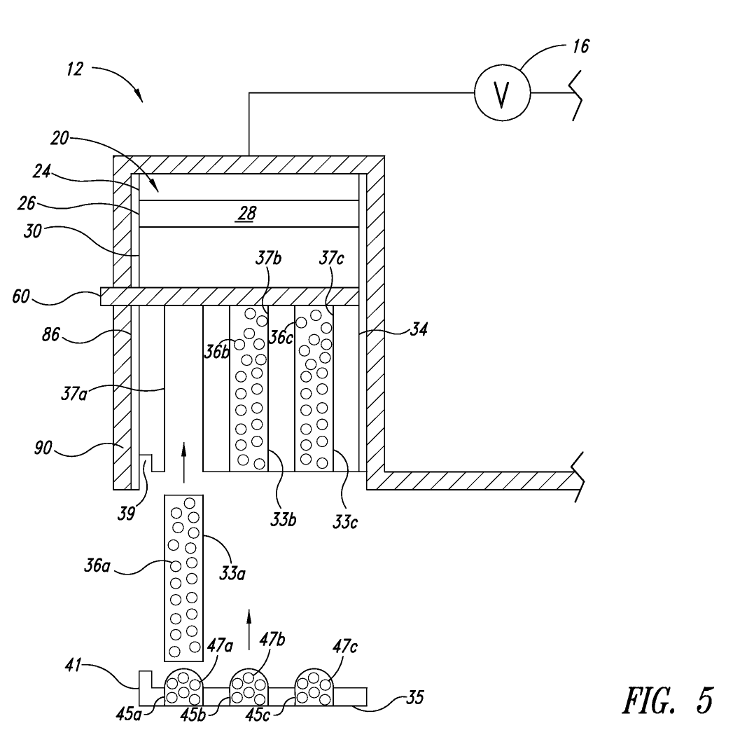

The blister pack 35 can be fixed in the iontophoretic device 10 or as shown in the illustrated embodiment of FIG. 5, the blister pack 35 can be in cartridge form insertably and/or removably secured in the iontophoretic device 10. For example, when the retaining structure 34 is also in cartridge form, the blister pack 35 can be replaced with the retaining structure 34 to replenish hydrating agent 47 and active agents 36. As shown in a partially exploded view in FIG. 5, retaining structure 34 may comprise receptacles 37a-37c (collectively 37) in which active agent reservoirs 33a-33c can be insertably and/or removably secured. FIG. 5 shows one of the active agent reservoirs 33a positioned for insertion into a receptacle 37a. The active agent reservoirs 33 may either be prepackaged with active agents 36 or be injected or otherwise loaded with active agents 36 upon or prior to use.

FIG. 5 shows one illustrated embodiment with the blister pack 35 awaiting to be insertably and/or removably secured between the retaining structure 34 and a biological interface (not shown). In such an embodiment, the blister pack 35 can also serve as an outer sealing liner or release liner or both. The blister pack 35 may further comprise at least one aligning mechanism 41 that can be complimentary to at least one guide element 39 of the retaining structure 34 to allow the blister pack 35 to be selectively positionable with respect to the receptacles 37 to hydrate selected ones of the active agents for use. In other embodiments, the guide element may be in any other portion of the active electrode 12. In yet other embodiments, the retaining structure 34 and blister pack 35 may be coupled as one cartridge removably secured in the iontophoretic device 10.

In still other embodiments, the blisters 45 may include active agents 36 or both active agents 36 and hydrating agents 47, allowing the active agent reservoirs 33 to be selectively loaded prior to use. In still other embodiments blister pack 35 may be adjacent or at least proximate to an outer ion selective membrane including distinct regions that retain active agents 36. In these embodiments, allowing active agents 36 to be in dehydrated form, retaining structure 34 cartridges can be prepackaged and provided with an iontophoretic device 10 that receives retaining structure 34 and blister pack 35 cartridges. In other embodiments, it may be desired to compose the further active agent 42 from one or more of the stored active agents 36, 40. In these embodiments, only those blisters containing the desired active agents can be pressed and broken to selectively load the further active agent 42.

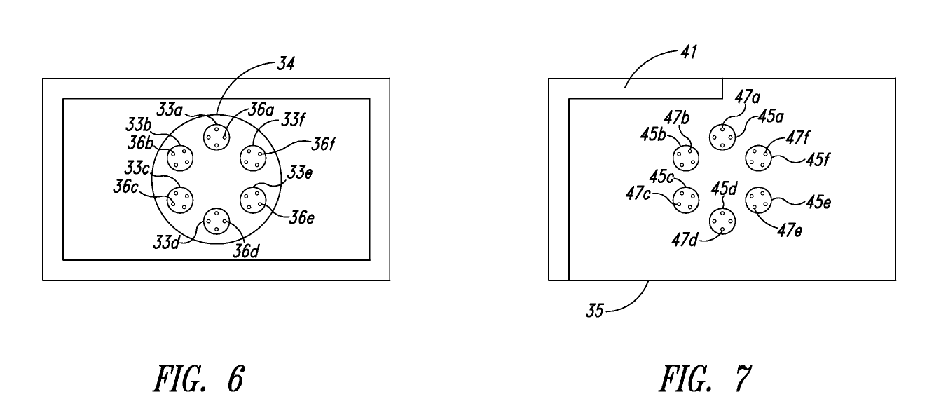

FIG. 6 is a cross sectional view of a retaining structure 34 in an active electrode assembly. As shown in FIG. 6, six active agent reservoirs 33a-33f (collectively 33) are spatially separated laterally from one another in a plane that is approximately parallel to a contact face 43 of the active electrode assembly 12 (shown in FIG. 4). Other embodiments may include a greater or lesser number of active agent reservoirs 33, and/or different lateral spacing patterns of the active agent reservoirs 33.

FIG. 7 shows an exemplary embodiment of the blister pack 35 including six blisters 45a-45f (collectively 45), agents 47a-47f (collectively 47) (e.g., hydrating and/or active agents), and an optional aligning mechanism 41. Agents 47a-47f may each be the same composition in some embodiments, or in other embodiments, they may each be distinct compounds or elements. Optional aligning mechanism 41 can align agents 47a-47f adjacent or at least proximate to respective ones of the active agent reservoirs 33a-33f. Prior to use, some or all of the agents 47 may be released by selectively breaking blisters 45 to hydrate and/or transfer active agents 36 through a biological interface (not shown).

The blister pack 35 may also aid the flow of active agent transfer through electroosmotic flow. During iontophoresis, the electromotive force across the electrode assemblies, as described, leads to a transfer of charged active agent molecules, as well as ions and other charged components, through the biological interface 18 into the biological tissue. This transfer may lead to an accumulation of active agents, ions, and/or other charged components within the biological tissue beyond the interface. During iontophoresis, in addition to the transfer of charged molecules in response to repulsive forces, there is also an electroosmotic flow of solvent (e.g., water) through the electrodes and the biological interface 18 into the tissue. In certain embodiments, the electroosmotic solvent flow enhances migration of both charged and uncharged molecules. Enhanced transfer via electroosmotic solvent flow may occur particularly with increasing size of the molecule.

In certain embodiments, the active agent may be a higher molecular weight molecule. In certain aspects, the molecule may be a polar polyelectrolyte. In certain other aspects, the molecule may be lipophilic. In certain embodiments, such molecules may be charged, may have a low net charge, or may be uncharged under the conditions within the active electrode. In certain aspects, such active agents may transfer poorly under the iontophoretic repulsive forces, in contrast to the transfer of small more highly charged active agents under the influence of these forces. These higher molecular active agents may thus be carried through the biological interface into the underlying tissues primarily via electroosmotic solvent flow. In certain embodiments, the high molecular weight polyelectrolytic active agents may be proteins, polypeptides or nucleic acids.

The above description of illustrated embodiments, including what is described in the Abstract, is not intended to be exhaustive or to limit the claims to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to other agent delivery systems and devices, not necessarily the exemplary iontophoresis active agent system and devices generally described above. For instance, some embodiments may omit some structures, may include additional structures, or both. For example, some embodiments may include a control circuit or subsystem to control a voltage, current or power applied to the active and counter electrode elements 24, 68. Also for example, some embodiments may include an interface layer interposed between the outermost ion selective membrane 38, 80 and the biological interface 18. Some embodiments may comprise additional ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240,995, herein incorporated in their entirety by reference. Further examples of such hydrogels are disclosed in U.S. Patent Application Nos. 2004/166147; 2004/105834; and 2004/247655, herein incorporated in their entirety by reference. Product brand names of various hydrogels and hydrogel sheets include CORPLEX™ by Corium; TEGAGEL™ by 3M; PURAMATRIX™ by BD; VIGILON™ by Bard; CLEARSITE™ by Conmed Corporation; FLEXIGEL™ by Smith & Nephew; DERMA-GEL™ by Medline; NU-GEL™ by Johnson & Johnson; and CURAGEL™ by Kendall, or acrylhydrogel films available from Sun Contact Lens Co., Ltd.

The various embodiments discussed above may advantageously employ various microstructures, for example microneedles. Microneedles and microneedle arrays, their manufacture, and use have been described. Microneedles, either individually or in arrays, may be hollow; solid and permeable; solid and semi-permeable; or solid and non-permeable. Solid, non-permeable microneedles may further comprise grooves along their outer surfaces. Microneedle arrays, comprising a plurality of microneedles, may be arranged in a variety of configurations, for example rectangular or circular. Microneedles and microneedle arrays may be manufactured from a variety of materials, including silicon; silicon dioxide; molded plastic materials, including biodegradable or non-biodegradable polymers; ceramics; and metals. Microneedles, either individually or in arrays, may be used to dispense or sample fluids through the hollow apertures, through the solid permeable or semi-permeable materials, or via the external grooves. Microneedle devices are used, for example, to deliver a variety of compounds and compositions to the living body via a biological interface, such as skin or mucous membrane. In certain embodiments, the active agent compounds and compositions may be delivered into or through the biological interface. For example, in delivering compounds or compositions via the skin, the length of the microneedle(s), either individually or in arrays, and/or the depth of insertion may be used to control whether administration of a compound or composition is only into the epidermis, through the epidermis to the dermis, or subcutaneous. In certain embodiments, microneedle devices may be useful for delivery of high-molecular weight active agents, such as those comprising proteins, peptides and/or nucleic acids, and corresponding compositions thereof. In certain embodiments, for example wherein the fluid is an ionic solution, microneedle(s) or microneedle array(s) can provide electrical continuity between a power source and the tip of the microneedle(s). Microneedle(s) or microneedle array(s) may be used advantageously to deliver or sample compounds or compositions by iontophoretic methods, as disclosed herein. In certain embodiments, for example, a plurality of microneedles in an array may advantageously be formed on an outermost biological interface-contacting surface of an iontophoresis device. Compounds or compositions delivered or sampled by such a device may comprise, for example, high-molecular weight active agents, such as proteins, peptides and/or nucleic acids.

In certain embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; an active agent reservoir having an active agent solution that is in contact with the first electrode member and to which is applied a voltage via the first electrode member; a biological interface contact member, which may be a microneedle array and is placed against the forward surface of the active agent reservoir; and a first cover or container that accommodates these members. The counter electrode assembly includes the following: a second electrode member connected to a negative electrode of the power source; an electrolyte reservoir that holds an electrolyte that is in contact with the second electrode member and to which voltage is applied via the second electrode member; and a second cover or container that accommodates these members.

In certain other embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; a first electrolyte reservoir having an electrolyte that is in contact with the first electrode member and to which is applied a voltage via the first electrode member; a first anion-exchange membrane that is placed on the forward surface of the first electrolyte reservoir; an active agent reservoir that is placed against the forward surface of the first anion-exchange membrane; a biological interface contacting member, which may be a microneedle array and is placed against the forward surface of the active agent reservoir; and a first cover or container that accommodates these members. The counter electrode assembly includes the following: a second electrode member connected to a negative electrode of the power source; a second electrolyte reservoir having an electrolyte that is in contact with the second electrode member and to which is applied a voltage via the second electrode member; a cation-exchange membrane that is placed on the forward surface of the second electrolyte reservoir; a third electrolyte reservoir that is placed against the forward surface of the cation-exchange membrane and holds an electrolyte to which a voltage is applied from the second electrode member via the second electrolyte reservoir and the cation-exchange membrane; a second anion-exchange membrane placed against the forward surface of the third electrolyte reservoir; and a second cover or container that accommodates these members.

Certain details of microneedle devices, their use and manufacture, are disclosed in U.S. Pat. Nos. 6,256,533; 6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; 6,939,311; all of which are incorporated herein by reference in their entirety. Some or all of the teaching therein may be applied to microneedle devices, their manufacture, and their use in iontophoretic applications.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: Japanese patent application Serial No. H03-86002, filed Mar. 27, 1991, having Japanese Publication No. H04-297277, issued on Mar. 3, 2000 as Japanese Patent No. 3040517; Japanese patent application Serial No. 11-033076, filed Feb. 10, 1999, having Japanese Publication No. 2000-229128; Japanese patent application Serial No. 11-033765, filed Feb. 12, 1999, having Japanese Publication No. 2000-229129; Japanese patent application Serial No. 11-041415, filed Feb. 19, 1999, having Japanese Publication No. 2000-237326; Japanese patent application Serial No. 11-041416, filed Feb. 19, 1999, having Japanese Publication No. 2000-237327; Japanese patent application Serial No. 11-042752, filed Feb. 22, 1999, having Japanese Publication No. 2000-237328; Japanese patent application Serial No. 11-042753, filed Feb. 22, 1999, having Japanese Publication No. 2000-237329; Japanese patent application Serial No. 11-099008, filed Apr. 6, 1999, having Japanese Publication No. 2000-288098; Japanese patent application Serial No. 11-099009, filed Apr. 6, 1999, having Japanese Publication No. 2000-288097; PCT patent application WO 2002JP4696, filed May 15, 2002, having PCT Publication No WO03037425; U.S. patent application Ser. No. 10/488970, filed Mar. 9, 2004; Japanese patent application 2004/317317, filed Oct. 29, 2004; U.S. provisional patent application Ser. No. 60/627,952, filed Nov. 16, 2004; Japanese patent application Serial No. 2004-347814, filed Nov. 30, 2004; Japanese patent application Serial No. 2004-357313, filed Dec. 9, 2004; Japanese patent application Serial No. 2005-027748, filed Feb. 3, 2005; Japanese patent application Serial No. 2005-081220, filed Mar. 22, 2005; U.S. Provisional Patent Application No. 60/722,136 filed Sep. 30, 2005; U.S. Provisional Patent Application No. 60/754,688 filed Dec. 29, 2005; U.S. Provisional Patent Application No. 60/755,199 filed Dec. 30, 2005; and U.S. Provisional Patent Application No. 60/755,401 filed Dec. 30, 2005.

As one skill in the relevant art would readily appreciate, the present disclosure comprises methods of treating a subject by any of the compositions and/or methods described herein.

Aspects of the various embodiments can be modified, if necessary, to employ systems, circuits and concepts of the various patents, applications and publications to provide yet further embodiments. While some embodiments may include all of the membranes, reservoirs and other structures discussed above, other embodiments may omit some of the membranes, reservoirs or other structures. Still other embodiments may employ additional ones of the membranes, reservoirs and structures generally described above. Even further embodiments may omit some of the membranes, reservoirs and structures described above while employing additional ones of the membranes, reservoirs and structures generally described above. Even further embodiments may omit some of the membranes, reservoirs and structures described above while employing additional ones of the membranes, reservoirs and structures generally described above.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An iontophoresis device operable to deliver active agents to a biological interface of a biological entity, comprising:
an active electrode assembly, the active electrode assembly including a contact face exposed on an exterior of the active electrode to be proximate to a biological interface when in use, an active electrode element operable to apply a first electrical potential, a first active agent reservoir capable of storing a first active agent, at least a second active agent reservoir capable of storing a second active agent, an outermost ion selective membrane exposed to the exterior of the iontophoresis device to form an interface with the biological interface, the outermost ion selective membrane substantially permeable by ions having a first polarity that matches a polarity of the first and the second active agents, and substantially impermeable by ions of a second polarity, opposite the first polarity, at least a portion of the first and second active agent reservoirs formed in the outermost ion selective membrane, the second active agent reservoir spaced laterally in a plane approximately parallel to the contact face from the first active agent reservoir, at least the first and the second active agent reservoirs positioned with respect to the active electrode element to each actively transfer at least some of the first and the second active agents from the iontophoresis device to the biological interface in response to application of the first electrical potential; and
a counter electrode assembly spaced laterally from the active electrode assembly, the counter electrode assembly including a counter electrode element operable to apply a second electrical potential, the second electrical potential being different from the first electrical potential.

2. The iontophoresis device of claim 1 wherein the active electrode assembly further includes an electrolyte positioned between the active electrode element and the first and the second active agent reservoirs, and an inner ion selective membrane positioned between the electrolyte and the first and the second active agent reservoirs, the inner ion selective membrane selectively substantially permeable by ions having the second polarity and substantially impermeable by ions having the first polarity.

3. The iontophoresis device of claim 2 wherein the active electrode assembly further includes an inner sealing liner withdrawalably positioned between the electrolyte and the first and the second active agent reservoirs.

4. The iontophoresis device of claim 1, further comprising:
an outer release liner covering the first and the second active agents prior to use.

5. The iontophoresis device of claim 1 wherein the active electrode assembly further includes the first active agent stored in the first active agent reservoir and the second active agent stored in the second active agent reservoir.

6. The iontophoresis device of claim 5 wherein the first active agent is a first antigen and the second active agent is a second antigen, different from the first antigen.

7. The iontophoresis device of claim 1 wherein the active electrode assembly, further includes at least a third active agent reservoir capable of storing a third active agent, the third active agent reservoir spaced laterally in the plane approximately parallel to the contact face from the first and the second active agent reservoirs, the third active agent reservoir positioned with respect to the active electrode element to actively transfer at least some of the third active agent from the iontophoresis device to the biological interface in response to application of the first electrical potential.

8. The iontophoresis device of claim 7 wherein the first, the second and the third active agents each have the first polarity.

9. An active agent delivery system operable to deliver active agents to at least two distinct areas on a biological interface, the active agent delivery system, comprising:
an active electrode element operable to provide a first electrical potential; and
a retaining structure having at least two receptacles, each of the receptacles configured to securely receive a respective active agent reservoir, the receptacles spaced laterally with respect to each other to overlie respective ones of the distinct areas on the biological surface when the active agent delivery system is in use; each of the receptacles at least partially underlying the active electrode element.

10. The active agent delivery system of claim 9, further comprising:
a first active agent reservoir configured to insertably secure within a first one of the receptacles; and
at least a second active agent reservoir configured to insertably secure within a second one of the receptacles.

11. The active agent delivery system of claim 10, further comprising:
at least a first active agent of a first polarity stored in the first active agent reservoir and substantially retained therein in the absence of an electromotive force or current and transferred outwardly in the presence of an electromotive force or current; and
at least a second active agent of the first polarity stored in the second active agent reservoir and substantially retained therein in the absence of an electromotive force or current and transferred outwardly in the presence of an electromotive force or current.

12. The active agent delivery system of claim 11 wherein the first and the second active agents are in dehydrated form prior to use.

13. The active agent delivery system of claim 12, further comprising:
a blister pack including at least two blisters of a hydrating agent, the blisters reputable to hydrate the first and the second active agents for use.

14. The active agent delivery system of claim 13 wherein the blisters are selectively reputable to hydrate selected ones of at least the first and the second active agents for use.

15. The active agent delivery system of claim 13 wherein the blister pack is positionable with respect to the receptacles.

16. The active agent delivery system of claim 13 wherein the blister pack is selectively positionable with respect to the receptacles to hydrate selected ones of at least the first and the second active agents for use.

17. The active agent delivery system of claim 13 wherein the retaining structure and blister pack are removably secured in place.

18. The active agent delivery system of claim 10, further comprising:
a blister pack including at least two blisters, each of the blisters holding a respective hydrating agent and a respective active agent, the blisters reputable to hydrate and load the active agents in the first and the second active agent reservoirs for use.

19. The active agent delivery system of claim 18 wherein the active agent in a first one of the blisters is different from the active agent in a second one of the blisters.

20. The active agent delivery system of claim 18 wherein the active agent in a first one of the blisters is a first antigen, and wherein the active agent in a second one of the blisters is a second antigen.

21. The active agent delivery system of claim 9, further comprising:
an electrolyte positioned between the active electrode element and the active agent receptacles.

22. The active agent delivery system of claim 9, further comprising:
an outermost ion selective membrane positioned to contact the biological interface when the active agent delivery system is in use.

23. An active agent delivery system, comprising:
an active electrode element operable to provide an electromotive force or current;
an outer ion selective membrane having an outer surface and at least two distinct regions laterally spaced from one another across the outer surface, each of the distinct regions having pores; and
at least two active agents of a first polarity cached within the pores of respective ones of the distinct regions of the ion selective membrane and substantially retained therein in the absence of the electromotive force or current and transferred outwardly from the ion selective membrane in the presence of the electromotive force or current.

24. The active agent delivery system of claim 23 wherein the outer ion selective membrane further has at least a third distinct region laterally spaced from the other distinct regions across the outer surface, and further comprising:
at least a third active agent of the first polarity cached within the pores of the third distinct region and substantially retained therein in the absence of the electromotive force or current and transferred outwardly from the ion selective membrane in the presence of the electromotive force or current.

25. The active agent delivery system of claim 24, further comprising:
a blister pack including at least two blisters of a hydrating agent, the blisters reputable to hydrate the active agents for use.

26. The active agent delivery system of claim 25 wherein the blisters are selectively reputable to hydrate selected ones of the active agents for use.

27. The active agent delivery system of claim 26 wherein the blister pack is positionable with respect to the distinct portions of the outmost ion selective membrane.

28. The active agent delivery system of claim 26 wherein the blister pack is selectively positionable with respect to the receptacles to hydrate selected ones of the active agents for use.

29. The active agent delivery system of claim 24, further comprising:
a blister pack including at least two blisters, each of the blisters holding a respective hydrating agent and a respective active agent, the blisters reputable to hydrate and load the active agents in the pores of the distinct regions of the ion selective membrane prior to use.

30. The active agent delivery system of claim 24 wherein the outmost ion selective membrane is an ion exchange membrane, the pores of the distinct regions containing an ion exchange material.
